(19) United States
(12) Patent Application Publication
(10) Pub. No.: US 2004/0161793 A1
(43) Pub. Date: Aug. 19, 2004

(54) Method for analyzing inhibition of ftsz polymerization using fluorescence assay technique

(76) Inventor: Dorina Trusca, West Orange, NJ (US)

Correspondence Address: MERCK AND CO INC, P O BOX 2000, RAHWAY, NJ, 070650907

(21) Appl. No.: 10/483,300

(22) PCT Filed: Aug. 6, 2002
(86) PCT No.: PCT/US02/27119

(30) Foreign Application Priority Data
Aug. 10, 2001 (US) 60311409

(51) Int. Cl. G01N 33/53

(52) U.S. Cl. 435/7.1

(57) ABSTRACT

A method for identifying chemical inhibitors of FtsZ polymerization using a direct fluorescence detection technique. The technique is based on the physical separation of fluorescently-labeled polymers of FtsZ from monomeric forms. This invention has both research and clinical applications in the identification of inhibitors of FtsZ polymerization as potential antibacterial agents.

### BACKGROUND OF THE INVENTION

Bacterial cell division is an essential process that maintains bacterial cell viability in infection and disease states. FtsZ plays a central role in bacterial cell division. Polymerization of the FtsZ bacterial protein forms a required structural element at the site of cell division. Therefore, inhibition of FtsZ polymerization prevents cell division, resulting in bacterial cell death. See D. Trusca, Journal of Bacteriology August 1998, Vol.180, No.15, p. 3946-3953; Yu, X. C. et al., EMBO J. 1997 16:5455-5463 and Erickson H P, et al., J. Cell Biol. 1996 135:5-8.

There has been extended interest in the development of a sensitive high-throughput assay to identify inhibitors of FtsZ polymerization. Existing methods of analyzing FtsZ polymerization include sedimentation and turbidimetric assay techniques (see Lee Y. C., et al., J Neurobiol 1974; 5(4): 317-30; Gaskin F., et al Ann N Y Acad Sci 1975 Jun. 30; 253:133-46; Hoebeke J, et al., Life Sci 1975 Aug. 15;17(4):591-5; Mukherjee A, et al., EMBO J 1998 Jan. 15;17(2):462-9; and

Bramhill D, et al. Proc Natl Acad Sci USA 1994 Jun. 21; 91(13): 5813-7). The sedimentation assay method provides accurate quantitative data pertaining to FtsZ polymerization, but is a slow method that cannot be scaled up for high-throughput screening. The turbidimetric assay is also insufficient, because the technique is insensitive at low concentrations of protein. Thus, there remains a need for a sensitive, rapid assay that can be translated into high-throughput screening format. The present invention relates to an assay that uses properties of fluorescence to screen for compounds that modulate the polymerization activity of FtsZ. In particular, an assay for rapidly screening compounds that inhibit FtsZ polymerization is provided. The compounds identified through this assay may be useful as antibiotic agents.

### SUMMARY OF THE INVENTION

The present invention relates to a method for screening compounds to identify modulators of the polymerization activity of FtsZ bacterial protein. In particular, an assay for rapidly screening compounds that inhibit FtsZ polymerization is provided. The invention further relates to an assay that is sensitive at low concentrations of protein.

### DETAILED DESCRIPTION OF THE INVENTION

A first embodiment of this invention is a method of analyzing the modulating effect of a compound on the polymerization activity of FtsZ proteins, comprising the steps of:

(a) combining in a plate containing a plurality of wells a mixture of wild-type FtsZ (Zwt) and fluorescently-labeled mutant FtsZT65C (ZFl) with the compound;

(b) initiating the polymerization reaction;

(c) incubating the mixture;

(d) centrifugation to separate polymeric proteins of wild-type FtsZ and fluorescently-labeled mutant FtsZT65C from monomeric proteins; and

(e) quantifying the amount of fluorescently-labeled mutant FtsZT65C polymeric protein retained on filter membranes.

A sub-embodiment of the method as recited above is wherein Step (a) is done by adding the compound to the plate which contains 1 μM wild-type FtsZ and 1 μM fluorescently-labeled mutant FtsZT65C, said plate having a 0.2 μm low protein binding Nylon membrane mounted to it, and containing 100 μl/well buffer containing 100 mM Tris(hydroxymethyl)aminomethane (Tris-Cl), at a pH of about 7.3 to about 7.6, preferably about 7.4, 1.5 mM Magnesium Acetate (MgAc), 67.5 mM Potassium Chloride (KCl), 1 mM Guanosine 5′-triphosphate (GTP), and 5 mM Calcium Chloride (CaCl₂).

A sub-embodiment of the method as recited above is wherein the generation of the mixture of wild-type and fluorescently-labeled mutant FtsZT65C proteins, comprises:

(a) centrifuging stock solutions of wild-type FtsZ and fluorescently-labeled mutant FtsZT65C at 356,000×g(g=gravity) for about 20 minutes at about 4° C.;

(b) adding aliquots of each stock solution of wild-type FtsZ and fluorescently-labeled mutant FtsZT65C to a Coomassie reagent;

(c) calculating the protein concentrations of wild-type FtsZ and fluorescently-labeled mutant FtsZT65C as the average of three readings at optical density (O.D.) 595 nm, based on a Bovine Serum Albumin (BSA) standard curve; and

(d) adding appropriate volumes of wild-type FtsZ and fluorescently-labeled mutant FtsZT65C to the 96-well plate, to yield a final concentration of 1 μM for each.

Another sub-embodiment of the method as recited above is wherein the polymerization reaction of Step (b) is initiated by adding Diethylaminoethyl-Dextran (DEAE-dextran MW=500,000 Da) at a concentration of about 50 μg/ml.

An additional sub-embodiment of the method as recited above is wherein, the incubation in Step (c) is done at 37° C. for 15 minutes.

Still another sub-embodiment of the method as recited above is wherein, the centrifugation in Step (d) is done by centrifuging the 96-well plate at 25° C., 3,000 rpm (2,060×g)(g=gravity) for about 15 minutes.

Yet in another sub-embodiment of the method as recited above is wherein the method for quantifying the amount of fluorescently-labeled mutant FtsZT65C polymeric protein retained on filter membranes (i.e., method for quantifying the pharmaceutical effectiveness of a test compound to inhibit FtsZ polymerization) of Step (e), comprises the steps of:

(a) measuring the amount of fluorescence on each sample well surface using a fluorescence scanner to determine the degree of polymer retention in the filter;

(b) calculating the level of polymerization as percent inhibition generated by each compound relative to fluorescence given by a polymerization reaction without inhibitor and compared to reaction containing 2 μM final concentration SulA (a.k.a. SfiA) protein; and

(c) evaluating sample activity as slightly active (75-100% inhibition), moderately active (16-74% inhibition), active (0-15% inhibition), or inactive (>or =100% inhibition). The inhibition values given by 2 μM SulA in control reactions are between 0-15%.

A sub-embodiment of the first embodiment of this invention is the method as recited above wherein the construction of fluorescently-labeled mutant FtsZT65C comprises:

(a) cloning of wild-type FtsZ into a pET11a vector and expression in BL21(DE3) host cells;

(b) performing site-directed mutagenesis (QuikChange™ Site-Directed Mutagenesis Kit) to replace threonine residue at position 65 with cysteine, to generate pET11aFtsZT65C (FtsZT65C) mutant construct; and

(c) reacting pET11aFtsZT65C construct with 5-iodoacetamidofluorescein (5-IAF)(Molecular Probes) to produce fluorescently-labeled mutant FtsZT65C or ZFl.

A second embodiment of the invention relates to a method of analyzing the modulating effect of a compound on the polymerization activity of FstZ proteins, comprising the steps of:

(a) combining on a plate containing a plurality of wells fluorescently-labeled mutant FtsZT65C with the compound;

(b) initiating the polymerization reaction;

(c) incubating the mixture;

(d) centrifugation to separate polymers of fluorescently-labeled mutant FtsZT65C from monomeric proteins; and

(e) quantifying the amount of fluorescently-labeled polymers retained on filter membranes.

A sub-embodiment of the method as recited above is wherein combining Step (a) is done by adding the compound to the plate containing 1 μM fluorescently-labeled mutant FtsZT65C, said plate having a 0.2 μm low protein binding Nylon membrane mounted to it, and containing 100 μl/well buffer containing 100 mM Tris-Cl, pH of about 7.3 to 7.6, preferably 7.4, 1.5 mM MgAc, 67.5 mM KCl, 1 mM GTP, and 5 mM CaCl₂.

Another sub-embodiment of the method as recited above is wherein the polymerization reaction of Step (b) is initiated by adding DEAE-dextran at a concentration of about 50 μg/ml.

In an additional sub-embodiment of the method as recited above the incubation in Step (c) is done at 37° C. for 15 minutes.

Still in another sub-embodiment of the method as recited above the centrifugation in Step (d) is done by centrifuging the 96-well plate at 25° C., 3,000 rpm (2,060×g)(g=gravity) for about 15 minutes.

Yet in another sub-embodiment of the method as recited above the method for quantifying the pharmaceutical effectiveness of a test compound to inhibit FtsZ polymerization of Step (e), comprises the steps of:

(a) measuring the amount of fluorescence on each sample well surface using a fluorescence scanner to determine the degree of polymer retention in the filter;

(b) calculating the level of polymerization as percent inhibition generated by each compound relative to fluorescence given by a polymerization reaction without inhibitor and compared to reaction containing 2 μM final concentration SulA (a.k.a. SfiA) protein; and

(c) evaluating sample activity as slightly active (75-100% inhibition), moderately active (16-74% inhibition), active (0-15% inhibition), or inactive (>or =100% inhibition). The inhibition values given by 2 μM SulA in control reactions are between 0-15%.

The instant invention provides a methodology useful in quantifying pharmaceutical effectiveness of a test compound to inhibit FtsZ polymerization. The effectiveness of an inhibitory test compound or compounds can be evaluated by initiating the FtsZ polymerization reaction in the presence of the test compound, followed by the execution of the instant invention to quantify the effectiveness. In this invention, polymers are trapped into a filter membrane with 0.2 μm size pores and monomers are separated upon centrifugation.

Chemicals and reagents were obtained from Sigma, unless otherwise stated. DEAE-dextran (Sigma #9885, average molecular weight of 500,000 Da). GTP and Lysozyme were purchased from Roche Molecular Biochemicals, IPTG from National LabSource, 5-iodoacetamidofluorescein (5-IAF) from Molecular Probes, NZ amine, yeast and tryptone from Difco, Tris(2-carboxyethyl)phosphine (TCEP-HCl) from Pierce, black upper structure Nylon filter plates with 0.2 μm pore size from Nalgene Nunc International.

Restriction enzymes were purchased from New England Biolabs, DNA polymerases for PCR, and QuickChange™ Site-Directed Mutagenesis kit from Stratagene.

Primers were synthesized by The Midland Certified Reagent Company, Texas.

DEAE-Sepharose Fast Flow and Sephadex G-25 were purchased from Pharmacia.

Strains and plasmids: Expression host Escherichia coli strains BL21(DE3) (F ompT hsdSB (r_{B}-m_{B}-) gal dcm (DE3)) and pET11a Amp^{R} were obtained from Novagen.

The recombination-deficient strain DH5α (recAl), devoid of any T7 RNA polymerase, was used as initial cloning host for all subcloning and mutagenesis studies and was bought from Life Technologies-Gibco.

The wild type pET11a-ftsz vector was mutagenized to obtain the pET11a-ftsZT65C, using the QuickChange™ Site-Directed Mutagenesis kit and following the Stratagene protocol. The two primers used to introduce the mutation were 5′-CGGTAGCGGTATCTGCAAAGGACTGGGCGC-3′ and 5′-GCGCCCAGTCCTTTGCAGATACCGCTACCG-3′.

The new construct pET11a-ftsZT65C derivative was confirmed using fluorescent dideoxy terminator sequencing on an ABI377 DNA sequencing machine.

Both FtsZ wild-type gene (Merck # MB5571) and FtsZT65C gene (Merck # MB 5572) will be deposited with the American Type Culture Collection, whose address is 10801 University Blvd., Manassas, Va. 20110-2209, under ATCC # PTA-4508 and ATCC# PTA-4509, respectively.

The mixture of wild-type and fluorescently-labeled FtsZ refers to an equimolar mixture of wild-type FtsZ and FtsZT65C mutant labeled with 5-iodoacetamidofluorescein (5-IAF), whereby the fluorescently-labeled FtsZ mutant generates the assay signal (fluorescence units λex=488 nm, λem=535 nm), and possesses comparable GTPase activity and polymerization property with wild-type FtsZ.

The reaction mixture consists of 100 mM Tris-Cl, pH of about 7.3 to about 7.6, preferably about 7.4, 1.5 mM MgAc, 67.5 mM KCl, 1 mM GTP pH 7.2, 5 mM CaCl₂, 1 μM wild type FtsZ, 1 μM fluorescently-labeled mutant FtsZT65C and 50 μg/ml DEAE-dextran. It is preferred that the pH of GTP be neutral, at about 7.0-7.5.

For purposes of this invention, aliquots refer to 1-2 μl of wild-type and/or fluorescently-labeled FtsZ protein in 1 ml of 1:1 Coomassie reagent: water (Coomassie reagent was purchased from Pierce, Inc., as Pierce Coomassie Plus reagent.

The BSA (bovine serum albumin) standard curve consists of seven BSA concentrations, 0, 0.5, 1, 1.5, 2, 2.5, 3 μg of protein measured in 1 ml of 1:1 Pierce Coomassie Plus reagent: water.

Prior to the assay, a multiscreen plate containing 96 wells with black upper structure and mounted 0.2 μm low protein binding Nylon membrane (Nalgene Nunc International) is equilibrated with 200 μl/well washing buffer consisting of 100 mM Tris-Cl, pH of about 7.3 to about 7.6, preferably about 7.4, 67.5 mM KCl, 1.5 mM MgAc and 0.1% Tween, followed by centrifugation at RT, 3,000 rpm (2,060×g) for 15 minutes.

The fluorescence is measured by inverting the centrifuged microtiter plate and scanning the 0.2 μm nylon filter membrane contained in each sample well using, for example, a STORM 860 fluorescence scanner (Molecular Dynamics) equipped with blue filter for fluorescence at 800 volts (V).

The SulA protein positive control refers to the sulA gene product in E. coli that is induced during the SOS response. FtsZ is a target of the SulA protein, and elevated levels of SulA lead to inhibition of cell division, filamentation, and subsequent cell death. (Huisman, O., et al., Nature 1981 290:797-99; Huisman, O., et al., Proc. Natl. Acad. Sci. USA. 1984 81(14): 4490-4; Bi E., et al., J. Bacteriol. 1993 175: 1118-25; and D. Trusca, et al., Journal of Bacteriology August 1998, Vol.180, No.15, p. 3946-3953). SulA at a final concentration of 2 μM is included as a positive control in 3 wells of each microtiter plate.

The process of this invention can be understood further by the following example, which does not constitute a limitation of the invention.

### EXAMPLE 1

Step A: Combining Wild Type and Fluorescently-Labeled FtsZ with the Compound

Wild-type FtsZ [also referred to as Zwt (freezer stock concentration 65.6 μM)] and fluorescently-labeled mutant FtsZT65C [also referred to as ZFl (freezer stock concentration 42.8 μM)] are centrifuged individually at 356,000×g for 20 minutes, at 4° C. in a TLA-100.2 rotor in a Beckman TL-100 Ultracentrifuge, immediately prior to reaction assembly to remove any pre-existing polymers. A 105×master mix, for one plate, is assembled, consisting of the appropriate amounts of: Tris-Cl, pH of 7.3 to 7.6, preferably about 7.4, (1000 mM stock), MgAc (550 mM stock), KCl (2000 mM stock), GTP (123 mM stock, pH 7.2), and CaCl₂ (500 mM stock). Zwt (65.6 μM stock) and ZFl (42.8 μM stock) are added once the protein concentrations are measured following centrifugation of the frozen stocks, as recited above.

Step B: Initiating the Polymerization Reaction

Prior to addition of the reaction components, the microtiter plate is equilibrated with 200 μl/well wash buffer containing 100 mM Tris-Cl, pH of about 7.3 to 7.6, preferably about 7.4, 67.5 mM KCl, 1.5 mM MgAc, and 0.1% Tween-20. The washing step is followed by centrifugation at room temperature at 3,000 rpm (2,060×g) (g=gravity) for 15 minutes.

The master mix is transferred to the microtiter plate at 100 μl/well. Inhibitor compound is added to each reaction at a final concentration of about 2 μM to about 10 μM, or SulA protein is added at a final concentration of about 0, 0.0625, 0.125, 0.25, 0.5, 0.75, 1, 1.5, to about 2 μM (preferably 2 μM) as a positive control. To initiate the polymerization reaction, DEAE-dextran (1000 μg/ml stock) is added to each reaction. Final concentrations of reaction components are as follows:

Tris-Cl pH of about 7.3 to 7.6, preferably about 7.4, (100 mM), MgAc (1.5 mM), KCl (67.5 mM), GTP pH 7.2 (1 mM), Zwt (1M), ZFl (11M), CaCl₂ (5 mM) and DEAE-dextran (50 μg/ml). The microtiter plate is incubated at 37° C. for 15 minutes.

Step C: Centrifugation to Separate FtsZ Polymers from Monomeric Proteins

Following incubation, the plate is centrifuged at 25° C. for 15 minutes at 3,000 rpm (2,060×g)(g=gravity) to separate polymers of Zwt/ZFl from monomeric proteins. Filtrate is discarded.

Step D: Quantifying the Amount of Fluorescently-Labeled Polymers Retained on Filter Membranes

The microtiter plate with mounted nylon filter is inverted and entire well surfaces are scanned using the STORM 860 fluorescence scanner, equipped with a blue filter for fluorescence at 800 V.

Control wells per plate are as follows: no DEAE-dextran added (6 wells), DEAE-dextran added (5 wells), and both DEAE-dextran and SulA added (3 wells). Two empty wells on each microtiter plate provide the value for membrane background.

Data are calculated as % inhibition given by compounds relative to control reactions without inhibitor. Quantitation is expressed in Fluorescence Units (FU), and both plate and fluorescent mixture backgrounds are subtracted from the fluorescence readings prior to the percentage inhibition calculation. Sample activity is evaluated as follows: slightly active (75-100% inhibition), moderately active (16-74% inhibition), active (0-15% inhibition), or inactive (>or =100% inhibition).

The inhibition values given by 2 μM SulA in control reactions are between 0-15%.

What is claimed is:

1. A method of analyzing the modulating effect of a compound on the polymerization activity of FtsZ proteins, comprising the steps of:
(a) combining in a plate containing a plurality of wells a mixture of wild-type FtsZ (Zwt) and fluorescently-labeled mutant FtsZT65C (ZFl) with the compound;
(b) initiating the polymerization reaction;
(c) incubating the mixture;
(d) centrifugation to separate polymeric proteins of wild-type FtsZ and fluorescently-labeled mutant FtsZT65C from monomeric proteins; and
(e) quantifying the amount of fluorescently-labeled mutant FtsZT65C polymeric protein retained on filter membranes.

2. A method for screening for compounds that inhibit FtsZ polymerization, comprising the steps of:
(a) combining in a plate containing a plurality of wells a mixture of wild-type FtsZ (Zwt) and fluorescently-labeled mutant FtsZT65C (ZFl) with the compound;
(b) initiating the polymerization reaction;
(c) incubating the mixture;
(d) centrifugation to separate polymeric proteins of wild-type FtsZ and fluorescently-labeled mutant FtsZT65C from monomeric proteins; and,
(e) quantifying the amount of fluorescently-labeled mutant FtsZT65C polymeric protein retained on filter membranes.

3. A method according to claim 2 wherein combining Step (a) is done by adding the compound to the plate which contains 1 μM wild-type FtsZ and 1 mM fluorescently-labeled mutant FtsZT65C, said plate having a 0.2 μm low protein binding Nylon membrane mounted to it, and containing 100 μl/well buffer containing 100 mM Tris(hydroxymethyl)aminomethane (Tris-Cl), at a pH of about 7.3 to about 7.6, preferably about 7.4, 1.5 mM Magnesium Acetate (MgAc), 67.5 mM Potassium Chloride (KCl), 1 mM Guanosine 5′-triphosphate (GTP), and 5 mM Calcium Chloride (CaCl₂).

4. A method according to claim 3 wherein the pH is 7.4.

5. A method according to claim 2 wherein the plate contains 96 wells.

6. A method, according to claim 2, wherein the generation of the mixture of wild type and fluorescently-labeled mutant FtsZT65C proteins, comprises:
(a) centrifuging stock solutions of wild-type FtsZ and fluorescently-labeled mutant FtsZT65C at 356,000×g)(g=gravity) for about 20 minutes at about 4° C.;
(b) adding aliquots of each stock solution of wild type FtsZ and fluorescently-labeled mutant FtsZT65C to a Coomassie reagent;
(c) calculating the protein concentrations of wild type FtsZ and fluorescently-labeled mutant FtsZT65C as the average of three readings at optical density (O.D.) 595 nm, based on a Bovine Serum Albumin (BSA) standard curve; and
(d) adding appropriate volumes of wild-type FtsZ and fluorescently-labeled mutant FtsZT65C to the 96-well plate, to yield a final concentration of 1 μM for each.

7. A method, according to claim 2, wherein the procedure for initiating the polymerization reaction comprises wherein the polymerization reaction of Step (b) is initiated by adding DEAE-dextran at a concentration of about 50 μg/ml.

8. A method, according to claim 2, wherein the incubation in Step (c) is done at 37° C. for 15 minutes.

9. A method according to claim 2 wherein the centrifugation in Step (d) is done by centrifuging the plate at 25° C., 3,000 rpm (2,060×g)(g=gravity) for about 15 minutes.

10. A method according to claim 2, wherein the method for quantifying the amount of fluorescently-labeled mutant FtsZT65C polymeric protein retained on filter membranes (i.e., method for quantifying the pharmaceutical effectiveness of a test compound to inhibit FtsZ polymerization) of Step (e), comprises the steps of:
(a) measuring the amount of fluorescence on each sample well surface using a fluorescence scanner to determine the degree of polymer retention in the filter;
(b) calculating the level of polymerization as percent inhibition generated by each compound relative to fluorescence given by a polymerization reaction without inhibitor and compared to reaction containing 2 μM final concentration SulA (a.k.a. SfiA) protein; and
(c) evaluating sample activity as slightly active (75-100% inhibition), moderately active (16-74% inhibition), active (0-15% inhibition), or inactive (>or =100% inhibition). The inhibition values given by 21M SulA in control reactions are between 0-15%.

11. A method according to claim 2, wherein the construction of fluorescently-labeled mutant FtsZT65C comprises:
(a) cloning of wild-type FtsZ into a pET11a vector and expression in BL21(DE3) host cells;
(b) performing site-directed mutagenesis to replace threonine residue at position 65 with cysteine, to generate pET 1 aFtsZT65C mutant construct; and
(c) reacting pET11aFtsZT65C construct with 5-iodoacetamidofluorescein (5-IAF) to produce fluorescently-labeled mutant FtsZT65C.

12. A method for screening for compounds that inhibit FtsZ polymerization, comprising the steps of:
(a) combining the compound, 1 μM wild-type FtsZ and 1 μM fluorescently-labeled mutant FtsZT65C on a plate containing a plurality of wells, said plate having a 0.2 μm low protein binding Nylon membrane mounted to it, and containing 100 μl/well buffer containing 100 mM Tris(hydroxymethyl)aminomethane (Tris-Cl), at a pH of 7.4, 1.5 mM Magnesium Acetate (MgAc), 67.5 mM Potassium Chloride (KCl), 1 mM Guanosine 5′-triphosphate at a pH of 7.2, (GTP), and 5 mM Calcium Chloride (CaCl₂);
(b) initiating the polymerization reaction by adding DEAE-dextran at a concentration of about 50 μg/ml;
(c) incubating the mixture at 37° C. for 15 minutes;
(d) centrifugation at 25° C., 3,000 rpm (2,060×g)(g=gravity) for about 15 minutes to separate polymeric proteins of wild-type FtsZ and fluorescently-labeled mutant FtsZT65C from monomeric proteins; and,
(e) quantifying the amount of fluorescently-labeled mutant FtsZT65C polymeric protein retained on filter membranes by: (1) measuring the amount of fluorescence on each sample well surface using a fluorescence scanner to determine the degree of polymer retention in the filter; (2) calculating the level of polymerization inhibition given by each compound as percent relative to fluorescence generated by reactions without inhibitor, (which is 2 μM final concentration SulA (a.k.a. SfiA) protein); and (3) evaluating sample activity as slightly active (75-100% inhibition), moderately active (16-74% inhibition), active (0-15% inhibition), or inactive (>or =100% inhibition). The inhibition values given by 2 μM SulA in control reactions are between 0-15%.

13. A method for screening for compounds that inhibit FtsZ polymerization, comprising the steps of:
(a) combining on a plate containing a plurality of wells fluorescently-labeled mutant FtsZT65C with the compound;
(b) initiating the polymerization reaction;
(c) incubating the mixture;
(d) centrifugation to separate polymers of fluorescently-labeled mutant FtsZT65C from monomeric proteins; and
(e) quantifying the amount of fluorescently-labeled polymers retained on filter membranes.

14. A method according to claim 13 combining Step (a) is done by adding the compound to the plate which contains 1 μM fluorescently-labeled mutant FtsZT65C, said plate having a 0.2 μm low protein binding Nylon membrane mounted to it, and containing 100 μl/well buffer containing 100 mM Tris-Cl, pH of about 7.3 to 7.6, preferably 7.4, 1.5 mM MgAc, 67.5 mM KCl, 1 mM GTP, and 5 mM CaCl₂.

15. A method according to claim 13, wherein the polymerization reaction of Step (b) is initiated by adding DEAE-dextran at a concentration of about 50 μg/ml.

16. A method according to claim 13, wherein the incubation in Step (c) is done at 37° C. for 15 minutes.

17. A method according to claim 13, wherein the centrifugation in Step (d) is done by centrifuging the 96-well plate at 25° C., 3,000 rpm (2,060×g) for about 15 minutes.

18. A method according to claim 13, wherein the method for quantifying the amount of fluorescently-labeled mutant FtsZT65C polymeric protein retained on filter membranes (i.e., method for quantifying the pharmaceutical effectiveness of a test compound to inhibit FtsZ polymerization) of Step (e), comprises the steps of:
(a) measuring the amount of fluorescence on each sample well surface using a fluorescence scanner to determine the degree of polymer retention in the filter;
(b) calculating the level of polymerization as percent inhibition generated by each compound relative to fluorescence given by a polymerization reaction without inhibitor and compared to reaction containing 2 μM final concentration SulA (a.k.a. SfiA) protein; and
(c) evaluating sample activity as slightly active (75-100% inhibition), moderately active (16-74% inhibition), active (0-15% inhibition), or inactive (>or =100% inhibition). The inhibition values given by 21M SulA in control reactions are between 0-15%.
